(19) United States
(12) Patent Application Publication
(10) Pub. No.: US 2025/0064682 A1
(43) Pub. Date: Feb. 27, 2025

(54) AN INTELLIGENT DIGITAL MEDICINE STORAGE AND REMINDER DEVICE

(71) Applicants: Kush PRAJAPATI, Ahmedabad (IN); Dhaumil Dipakbhai PARMAR, Ahmedabad (IN); Harsh Yogeshbhai MANGUKIYA, Surat (IN)

(72) Inventors: Kush PRAJAPATI, Ahmedabad (IN); Dhaumil Dipakbhai PARMAR, Ahmedabad (IN); Harsh Yogeshbhai MANGUKIYA, Surat (IN)

(21) Appl. No.: 18/722,629

(22) PCT Filed: Dec. 20, 2022
(86) PCT No.: PCT/IB2022/062516

§ 371 Date: Jun. 21, 2024

(30) Foreign Application Priority Data
Dec. 21, 2021 (IN) 202123059667

(51) Int. Cl. A61J 7/04; G16H 20/13

(52) U.S. Cl. CPC A61J 7/049; A61J 7/0427; A61J 7/0454; G16H 20/13; A61J 2205/60

(57) ABSTRACT

The present invention relates to an intelligent digital medicine storage and reminder device (1). The intelligent digital medicine storage and reminder device (1) having an intelligent compartment (100) which comprises a LED (104), audio notification, visual signals and track medicine. The separate lock provided for the each compartment (100). The RFID detector (106) provided to identify the user once tapping RFID card and provide the time and schedule dosage of medicine. The QR code (201) provided which can be scanned by the mobile means to track schedule of medicine and to identify the user. For identifying the user the finger print sensor (307) provided. The intelligent compartment (100) eliminates the direct contact of the human hands with the medicine.

This application is the patent of addition of Indian Patent Application number 201921050118 filed on 5 Dec. 2019.

### FIELD OF THE INVENTION

The present invention relates to an intelligent digital medicine storage and reminder device. Particularly it relates to an intelligent digital device for storing and reminding medicines which are to be taken at different times or days and to remind the user to take particular medicines at specific times. It also discloses a device with a QR code located beside the touch display, can be scanned by mobile means. It helps to track the schedule of medicine and to identify the user for whom and how many medicines needs to consume at a particular time. It also relates to a device that can be automatically provides a reminder to the user through wireless connectivity. It also relates to the device being capable of interface with any communication device to schedule, generates, and communicates the relevant data to the caretaker or healthcare provider, or any concerned party.

### BACKGROUND OF THE INVENTION

Patient compliance or adherence describes the degree to which a patient correctly follows a medication schedule. Most commonly, it refers to medication or drug compliance. Medication compliance hence is described as the degree to which the patient follows the prescribed and dispensed regimen as intended by the prescriber and dispenser. Medicine compliance by the patient is a primary determinant of treatment success.

Noncompliance is a great problem faced by the health care system and a major obstacle to the effective delivery of health care. 2003 estimates from the World Health Organization indicated that only about 50% of patients with chronic diseases living in developed countries follow treatment recommendations with particularly low rates of adherence to therapies for asthma, diabetes, and hypertension.

Major barriers to compliance are thought to include the complexity of modern medication regimens where order, dose, and direction to take medicines are very crucial. Co-occurrence of multiple diseases further leads to non-compliance as concomitant medications are required. It also includes the inability to remember medication schedules because of age, poor mental health, and a busy work schedule. The problem of non-compliance arises in the case of chronic illness which requires therapy for a prolonged period of time and has a frequent dosing schedule.

Hence there is need to design a device to deal with different medication with the different dose which is to be taken at different time.

Devices and systems are already known for counting the medicines/tablets/pills/drugs and reminding the same to the patients at the prescribed time. One of which is a multi-compartment pillbox to help organize the task of taking the proper medication at the proper time. These compartments are designated AM, PM, or Breakfast, Lunch, Dinner, or some other set of designations, for instance, by time.

The compartments may be integral or may be individual small boxes that are retained in a day-frame so that each can be individually manipulated. Pill organizers typically may have seven of such daily four-compartment boxes, arranged according to the seven days of the week.

A patient or a patient's caregiver determines which medications are required for each day, and at what time of the day. The required pills are placed into the corresponding compartments, the compartments are closed and each day-set is put into the week-frame for safe-keeping. At a particular time, the patient or the patient's caregiver opens the appropriate compartment, removes the medication, and the patient consumes it. It is refilled with the proper medications at a particular interval.

Such manual medication systems are simple and inexpensive however have disadvantages related to loading and removal of medicine. Because of complex medication regimens, it may be complicated to ensure that the correct medication is placed in the compartment that corresponds to the correct time to take that medication. If the duplicate pill is placed or the pill is omitted, which may result in an overdose or underdose. This task is also complicated in case of older age and mental incapability. Turning to the dispensing disadvantages, a typical day-set contains four compartments, and a typical week-set contains seven day-sets, for a total of twenty-eight dose medication compartments. Further, a patient might take medicine from the wrong compartment. A patient may forget to take any medication at a prescribed time. It also relates to the lack of real-time remote visibility and data generation for caregivers or concerned parties to monitor compliance with the medication schedule.

As the technology evolved over time, many electronic automated and non-automated medicine storage and reminder devices are developed.

U.S. Pat. No. 564,612 discloses medication compliance, coordination, and dispensing system which comprises a cabinet that is centrally located, a free-standing main housing unit, and two rows of smaller individual units arranged on two opposite sides of the main unit and engageable with the main unit. Each of the smaller individual units functions independently to store and dispense medication.

U.S. Pat. No. 5,720,154 discloses an article dispensing system comprising a supply hopper to dispense different articles, a first buffer chamber having an outlet and an outlet door, means for counting and advancing articles from the supply hopper to the first buffer chamber, first means for moving the outlet door to release the articles from the first buffer chamber in response to a first predetermined condition, a second buffer chamber for receiving articles from the first buffer chamber, the second buffer chamber having an outlet and an outlet door; and second means for moving the door of the second buffer chamber to release the articles from the second buffer chamber in response to a second predetermined condition.

US20120006847 discloses an interactive medicine organizer which assists the individual who needs to organize or monitor the administration of one or more medications. It discloses an apparatus that comprises a circular dispenser body with thirty chambers divided into compartments for storage of the pill.

US2013002795 discloses systems and methods for ensuring compliance by a patient in taking scheduled medications. It instructs the user through visual and audio cues, such as the illumination of individual medication cups that are arrayed in accordance with a daily and weekly schedule in separate orifices within the dispenser body.

The problems associated with available medicine storage and reminder system are also that there was a need to manually extract the medicines 3-4 times a day and there is a need to fill the medicine box in every 7-14 days. Another problem associated with the available medicine dispenser system is that the medicines/pills cannot be stored too long as they are in contact with each other and are in contact with the external environment.

This will cause drug-to-drug interaction. A drug-drug interaction can delay, decrease, or enhance the absorption of either drug. This can decrease or increase the action of either or both drugs or cause adverse effects. Even if the medicine box has the ability to remember a schedule of medicine from day to day, the person or patient might take medicine from the wrong compartment. In circumstances where the medicine cannot be taken when the timer sounds, a person's entire medicine schedule may become delayed. Many existing devices neither monitor whether the medicine was taken, or at what time the medicine was taken nor communicate this data to the interested person. Also, most of these devices must be re-programmed on a daily basis. This re-programming introduces the opportunity to make errors and may be too difficult for the elderly. The available devices are also cumbersome to carry on a daily basis.

Another problem associated with the available medicine storage and reminder system is that the prior technologies are not operated automatically and not disclosing the intelligent compartment which being capable to lock each compartment separately. With the said arrangement, the particular compartment opened at the time of particular dosage.

So, it is desperately needed to introduce a compact, effective, digital, intelligent digital medicine storage and reminder device having intelligent compartment provided with a separate lock and also the device having a sensor, detectors, camera, touch display, and scanners. The device has an electrically as well as rechargeable battery-powered and is a digitally programmable device that can overcome all problems described above. Also, the artificial intelligence-based prediction for identifying which medicine not taken as per the schedule. The invention of the present invention is as described herein.

### OBJECT OF THE INVENTION

The principal object of the present invention is to provide an intelligent digital medicine storage and reminder device.

Another object of the present invention is to provide an intelligent digital medicine storage and reminder device with the features like wireless connectivity, reminder system, buzzer snooze button, SoS feature, and charging port.

Another object of the present invention is to provide an intelligent digital medicine storage and reminder device being capable to continuously remind the user or caretaker at a specific time of dosage of a specific medicine.

Another object of the present invention is to provide an intelligent digital medicine storage and reminder device that can easily detect and identify the user's RFID card.

Another object of the present invention is to provide an intelligent digital medicine storage and reminder device which can be accessed by fingerprint.

Another object of the present invention is to provide intelligent digital medicine storage and reminder device being capable to track the schedule of medicine and identify the user by QR code which can scan by mobile means.

Another object of the present invention is to provide intelligent digital medicine storage and reminder device being capable of an artificial intelligence-based prediction which can detect the consumption of medicine of user.

Another object of the present invention is to provide a feature to set medicine schedule for the user and also reminding user through different reminder indicators i.e. Led indication, buzzing alarm and particular dose of medicine need to take has shown on the display.

### SUMMARY OF THE INVENTION

The present invention is all about an intelligent digital medicine storage and reminder device.

The aspect of the present invention is an intelligent digital device for storing medicines which needs to be taken at different times or days and to remind the user to take particular medicines at specific times and also will notify the user/caretaker in case patient don't take medicines and can provide adherence report also remind the user when will medicines finish out.

The main aspect of the present invention is to provide an intelligent digital medicine storage and reminder device comprising:
a) an intelligent compartment having a housing being filled with medicine strips;
b) a door being connected to each compartment, each door openable for releasing the medicine at the time of dosing schedule;
c) a RFID detector located at the front side of the device;
d) a LED and buzzer being connected to each door to reminding the time of taking medicine to the user;
e) a touch display unit, said touch display unit being mounted on the device having the QR code which can be scanned by the mobile means to track schedule of medicine and to identify the user;
f) a microcontroller being capable to control the operation of the intelligent digital medicine storage and reminder device;
g) a control panel being capable to control the intelligent digital medicine storage and reminder device;
h) a battery, said battery supplies the demand power to the said device;
i) a finger print sensor being capable to identify the user finger and unlock the compartment at the scheduled time;
j) a smart camera;
characterised by,
said intelligent compartment provided with a separate lock; said separate lock provided for unlocking the particular compartment on prescribed medicine schedule; said RFID detector identify the user on tapping RFID card and provide the time and schedule dosage of medicine; said intelligent compartment append the customized pack of medicines as per patient prescription; said medicine storage and reminder device notify the user through LED blinking, audio notification and visual indication and track the medicine which is to be taken as per prescribed schedule and artificial intelligence based prediction to identify medicine which is not taken as per schedule.

Yet another aspect of the present invention is to provide the intelligent digital medicine storage and reminder device wherein the door being capable to open automatically at the time of particular dosage and the LED indicates medicine intelligent compartment along with buzzing of the buzzer till the door being open or pressing the snooze button.

Yet another aspect of the present invention is to provide the intelligent digital medicine storage and reminder device wherein the door closed after removal of the medicine from the intelligent compartment or pressing the snooze button, the device automatically records that the dose as a successfully administered dose.

Yet another aspect of the present invention is to provide touch display shows the real-time date and time, patient name, reminding pop-up access, health tips, emergency call service, editing and adding features and alert to the next dose, name of the medicine and doses, schedule time including wireless connectivity, battery status.

One more aspect of the present invention is to provide a RFID detector that identify the user on tapping the RFID card and being capable determine the time and schedule of the medicine for the user.

Yet another aspect of the present invention provides an intelligent digital storage and reminder device being capable to track medicine through artificial intelligence-based smart camera.

### BRIEF DESCRIPTION OF THE DRAWINGS

The above and other objects and features of the present invention will become apparent from the following description of the invention, when taken in conjunction with the accompanying drawings, in which:

FIG. 1 elaborated a diagram of an intelligent digital medicine storage and reminder device.

FIG. 2 schematic diagram of an intelligent digital medicine storage and reminder device.

FIG. 3 shows the schematic view of intelligent digital storage and reminder device.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Unless defined otherwise, all technical and scientific terms used herein have the same meaning as commonly understood by one of ordinary skill in the art to which the invention belongs.

The present invention overcomes the problem of an intelligent digital medicine storage and reminder device. The object, features, and advantages of the present invention will now be described in greater detail. Also, the following description includes various specific details and is to be regarded as merely exemplary. Accordingly, those of ordinary skill in the art will recognize that: without departing from the scope and spirit of the present disclosure and its various embodiments there may be any number of changes and modifications described herein.

It must also be noted that as used herein and in the appended claims, the singular forms “a”, “an,” and “the” include plural references unless the context clearly dictates otherwise. Although any systems and methods similar or equivalent to those described herein can be used in the practice or testing of embodiments of the present invention, the preferred, systems are now described.

The term “RFID” means Radio Frequency Identification refers to a wireless system comprised of two components tags and readers. The reader is a device to scan the QR code and said reader having one or more antennas being capable to emit the radio waves and receives radio waves back from the RFID tag.

As per main embodiment of the present invention, an intelligent digital medicine storage and reminder device (1) comprising an intelligent compartment (100) having a housing (101) being filled with medicine strips (102), a door (103) being connected to each compartment (100), each door (103) openable for releasing the medicine at the time of dosing schedule, a RFID detector (106) located at the front side of device (1), a LED (104) and buzzer (105) being connected to each door (103) to reminding the time of taking medicine to the user, a touch display (200) unit, said touch display (200) unit being mounted on the device having the QR code (201) which can be scanned by the mobile means to track schedule of medicine and to identify the user, a microcontroller (110) being capable to control the operation of the intelligent digital medicine storage and reminder device (1), a control panel (300) being capable to control the intelligent digital medicine storage and reminder device (1), a battery, said battery supplies the demand power to the said device, a finger print sensor (307) being capable to identify the user finger to finger lock and unlock the compartment (100) at the scheduled time, a smart camera, characterised by, said intelligent compartment (100) provided with a separate lock; said separate lock provided for unlocking the particular compartment (100) on prescribed medicine schedule; said RFID detector (106) identify the user on tapping RFID card and provide the time and schedule dosage of medicine; said intelligent compartment (100) append the customized pack of medicines as per patient prescription; said medicine storage and reminder device (1) notify the user through LED (104) blinking, audio notification and visual indication and track the medicine which is to be taken as per prescribed schedule and artificial intelligence based prediction to identify medicine which is not taken as per schedule.

As per one embodiment of the present invention is the door (103) being capable to open automatically at the time of a particular dosage and the LED (104) indicates a medicine intelligent compartment (100) along with the buzzing of the buzzer (105) till the door (103) being open or pressing the snooze button.

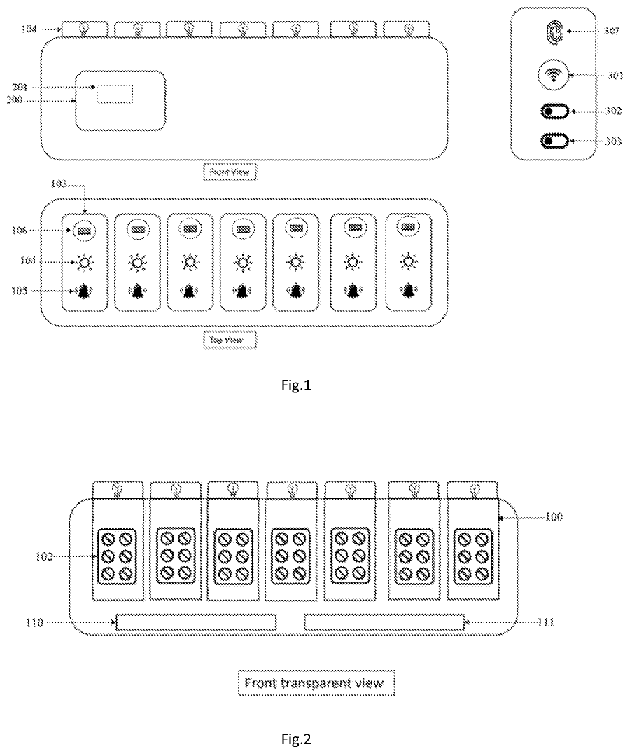

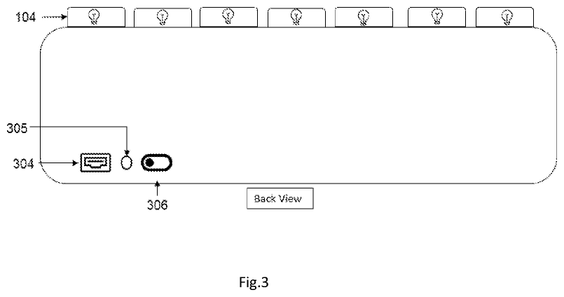

Referring to FIG. 1, FIG. 2, and FIG. 3 the intelligent digital medicine storage and reminder device (1) comprises an intelligent compartments (100) to hold medicines. Medicines can be tablets, capsules, pills, sachet, stick pack, mini-tablet, pellet, beads, spheres, spheroids, mini spheres, straw device (X-straw), and other formulations. As per one embodiment, the intelligent compartments (100) are provided to avoid direct contact with the medicines. The intelligent compartment (100) is provided with a separate lock and the separate lock is for unlocking the particular intelligent compartment (100) on the prescribed medicine schedule. Also, the intelligent compartment (100) appends to a customized package as per patient prescription. The medicine container can be prepared of any size as per the requirement of the user and it can be of small, medium, and large sizes. The small size of intelligent compartments (100) can hold about 25-30 small size units, 10-20 medium-size units, and 5-10 large size units of medicines. The medium size of intelligent compartments (100) can hold about 150-200 small size units, 90-120 medium-size units, and 70-80 large size units of medicines in the form of strips. For the large size of intelligent compartments (100), it can be designed and attached as per the requirement of hospitals or clinics. Medicines mentioned here mean strips of tablet or capsule or any other mentioned above. The intelligent compartment (100) comprises a compartment door (103). Each intelligent compartment (100) door (103) having reminder means such as LED (104) light, audio notification, and visual indications to remind the time of dosage to the user. The said LED (104), audio notification, and visual indication indicate to the user through which intelligent compartment (100) user has to take the medicine. The device (1) is compact and simple which can be carried easily.

As per one embodiment, the intelligent compartment (100) is having the size enough to store the whole strip (102) of medicines rather than emptying the individual medicines. This offers an advantage in comparison to existing devices where the patient has to empty all medicines and be filled in the container which results in exposure of medicines to the atmosphere and is prone to degradation before expiry. In the intelligent compartment (100), the user adding the existing pill-packed with respective medicines added in respective packages depending on their timings, the device (1) notifies the user to take the pill-packaged using LED (104) blinking, audio notifications & visual indication on display (200), and keep track of pill-packaged that have been taken and the remaining too.

As per the present invention, the intelligent compartment (100) is having enough space to place a small size strip (102) as such or can be placed by cutting the strip (102) into two parts without taking medicine out from its original packaging.

As per the one embodiment of the present invention, the RFID detector (106) being capable to identify the user on tapping RFID cards and provide the time and schedule dosage of medicine. The RFID detector (106) being capable to automatic identification and data capture which can automatically identify objects, collect data about them, and enter those data directly into the system. The RFID tag contains an integrated circuit and an antenna, which are used to transmit data to the RFID detector (106). When the user taps the RFID tag, the RFID detector (106) converts the radio waves to a more usable form of data, and information collected from the tags is then transferred through a communications interface to a system, where the data can be stored in a database and analysed the time and schedule of the medicine for the user.

As per one embodiment, the touch enable display (200) is a low-power consumption multi-color, black and white E-paper display, different Led display. E-paper display is eco-friendly and low power consuming display, this will also show the real-time date and time, patient name, reminding pop-up access, health tips, emergency call service, editing and adding features and alert to the next dose, name of the medicine & doses, schedule time including wireless connectivity, battery, Wi-Fi signals, battery level indicator which is the innovation in itself. It can also indicate to the user for which and how many medicines to take.

As per the one embodiment, the touch display having the QR code (201) which can be scanned by mobile means. The QR code (201) provided to track the schedule of medicine and to identify the user. The QR code (201) being mounted beside the touch display (200) on the device and the user can verify the dosage of medicine by scanning QR code (201) from mobile means.

As per the one embodiment of the present invention is provide an intelligent digital medicine storage and reminder device being capable to identify the user by fingerprint sensor (307). The intelligent digital medicine storage and reminder device (1) having a fingerprint sensor (307) beside the touch display (200) unit and the user can unlock the intelligent compartment (100) by the fingerprint.

Another embodiment of the present invention is the microcontroller (110) being capable to run the system through the program with the help of a microcontroller (110). The unit provides coordination among all components of the device depending on technology, some of the principal parts should work inside the microcontroller (110). The microcontroller (110) being connected to each component through the installed program and provides the result. The microcontroller (110) can be selected but not limited from raspberry pi, arduino, arm controller etc.

As per one embodiment, the application need to be installed in the microcontroller (110) to run the whole device mechanism. Said application based on machine learning, data analysis, and artificial intelligence to analysis.

The intelligent digital medicine storage and reminder device (1) comprises a buzzer (105) being capable to produce an audio signal at the pre-defined set time for indicating the time to take medicine. It also comprising snooze button (302) for the further reminding to the user.

The intelligent digital medicine storage and reminder device (1) comprises a wireless connectivity button (301) which is used to turn on the wireless connectivity for the connection in the device. The wireless connection used to set medicine timing and schedule. Wireless connection is also required to interface the device to the mobile means. The mobile means used to program medicine timing and schedule, maintains the records regarding medicine compliance. It also allows a person to direct notify the emergency number in time of emergency with the SoS button (303).

The intelligent digital medicine storage and reminder device (1) described herein being capable to driven by a centralized circuit. The printed circuit board comprises all modules and components mounted on it. The Modules comprise but are not limited to wireless connection module, wired connection module, Bluetooth module, Real-time circuit module. The wireless connection module capable of supporting 2G, 3G, 4G or 5G networks. The intelligent device comprises of battery (111) or a power supply to provide power to the digital automated medicine storage and reminder system. The entire device is the battery (111) operated. Battery (111) can be rechargeable or non-rechargeable. The battery can be Nickel Cadmium (NiCd) Battery, Nickel-Metal Hydride (NiMH) Battery, Lithium-Ion (Li-ion) Battery and Lithium-Ion Polymer (Li-ion polymer). In the case of a rechargeable battery (111), it can be charged using a charging port (304). The battery (111) is long life and can run the device for a long time without frequent charging. The battery life status is displayed in the display panel (200).

The embodiment of the present invention is to provide an intelligent medicine storage and reminder device (1) being capable to track the consumption of medicine based on artificial intelligence prediction. The artificial intelligence prediction in the intelligent medicine storage and reminder device (1) being capable to identify the dosage time, which dose needs to be taken at particular time, which dose remaining, which doses need to be taken in the future.

From the above, it can be readily observed that the intelligent digital medicine storage and reminder device of the present invention are user-friendly and can be effectively used to follow a prescribed regimen in a timely and proper manner to enhance compliance, and thus the overall well-being of as subject.

Another embodiment of the present invention, the intelligent digital medicine storage and reminder device (1) further configured to compute a count of the medicines stored in each of the medicine compartments. The intelligent digital medicine storage and reminder device (1) receive data representing the count of the medicines from each of the sensors installed in each of the medicine intelligent compartments (100). The sensors can be selected but not limited from magnetic sensor, photo sensor, IR sensor etc. Furthermore, the intelligent digital medicine storage and reminder device (1) may be further configured to transmit the count of the medicines to the display module, in an embodiment of the present invention.

As per one embodiment, the device (1) of the present invention, along with intergraded mobile application and web portal can also provide the report of the medicine adherence and scheduling.

As per one embodiment, the device is also equipped with a braille mechanism to make it user-friendly for blind people.

As per one embodiment, the device (1) also being capable to paired with wearable devices, smart speakers. The user needs to configure the intelligent digital medicine storage and reminder device (1) and it is ready to use through the wearable. This function is available for all users but is mainly beneficial to people with hearing impairment or deaf people. They can get alert in terms of vibration on the wearable.

As per one embodiment, all the data and reports can also be accessed through a web portal or mobile application.

The device of the present invention is helpful to many classes of end-users like patients, caretakers, hospitals, and private clinics.

Referring to FIG. 1 and FIG. 2, it shows that the housing (101) of the intelligent compartment (100) can be filled with the whole strip so the user does not fill the intelligent compartment (100) by unpacking the individual medicine manually. The intelligent compartment (100) comprises the door (103) through which the medicine is delivered at a specific time. The door (104) is automatically open at the specific time of dosage and the LED (104) blinking, audio notification, and visual indication and the buzzer (105) buzzing till the door is open. Hence, if the user forgets to take the medicine the LED (104) and buzzer (105) continuously pass the signal to the user. The control panel (300) comprising the wireless connectivity button (301), the buzzer snooze button (302) for the reminder, the SoS button (303) to call on the saved number in case of any emergency, and a charging port (304) to charge the device (1). Further, the RFID detector (106) is located at front side to identify the user on tapping RFID card and provide the time and schedule dosage of medicine. Also, the fingerprint lock (307) on the touch display being capable to identify the user's finger and unlock the intelligent compartment at the scheduled time. The QR code (201) on the touch display being capable to scan by mobile means to track the schedule of medicine and to identify the user.

Referring FIG. 3, shows that the LED (104) indication being connected to each door (103) to reminding the time of taking medicine to the user. The charging pot being capable for to charge the battery (111) in the intelligent digital medicine storage and reminder device (1). Also, the device on/off button (306) is available on an intelligent device, and the reset pin (305) being capable to reset the whole system.

### LIST OF REFERENCE NUMERALS

Intelligent digital medicine storage and reminder device (1)
Intelligent compartment (100)
Housing (101)
Medicine strips (102)
Door (103)
LED (104)
Buzzer (105)
RFID detector (106)
Microcontroller (110)
Battery (111)
Display (200)
QR code (201)
Control panel (300)
Wireless connectivity button (301)
Buzzer snooze button (302)
SoS button (303)
Charging port (304)
Reset pin (305)
Device on/off button (306)
Fingerprint sensor (307)

We claim:

1. An intelligent digital medicine storage and reminder device (1) comprising:
a) an intelligent compartment (100) having a housing (101) being filled with medicine strips;
b) a door (103) being connected to each compartment, each door (103) openable for releasing the medicine at the time of dosing schedule;
c) a RFID detector (106) located at the front side of the device;
d) a LED (104) and buzzer (105) being connected to each door (103) to reminding the time of taking medicine to the user;
e) a touch display (200) unit, said touch display (200) unit being mounted on the device (1) having the QR code (201) which can be scanned by the mobile means to track schedule of medicine and to identify the user;
f) a microcontroller (110) being capable to control the operation of the intelligent digital medicine storage and reminder device;
g) a control panel (300) being capable to control the intelligent digital medicine storage and reminder device;
h) a battery, said battery (111) supplies the demand power to the said device;
i) a finger print sensor (307) being capable to identify the user finger and unlock the compartment (100) at the scheduled time;
j) a smart camera;
characterised by,
said intelligent compartment (100) provided with a separate lock; said separate lock provided for unlocking the particular intelligent compartment (100) on prescribed medicine schedule; said RFID detector (106) identify the user on tapping RFID card and provide the time and schedule dosage of medicine; said intelligent compartment (100) append the customized pack of medicines as per patient prescription; said medicine storage and reminder device (1) notify the user through LED (104) blinking, audio notification and visual indication and track the medicine which is to be taken as per prescribed schedule and artificial intelligence based prediction to identify medicine which is not taken as per schedule.

2. The intelligent digital medicine storage and reminder device (1) as claimed in claim 1, wherein the door (103) being capable to automatically open at the time of particular dosage and the LED (104) indicates medicine intelligent compartment (100) along with the buzzing of the buzzer (105) till the door (103) being open or pressing the snooze button.

3. The intelligent digital medicine storage and reminder device (1) is claim as claimed 1, wherein the door (103) closed after removal of the medicine from the intelligent compartment (100) or pressing the snooze button, the device (1) automatically records that the dose as a successfully administered dose.

4. The intelligent digital medicine storage and reminder device (1) as claimed in claim 1, wherein the control panel (300) comprising a wireless connectivity button, a buzzer (105) snooze button, a SoS button (303) being capable to call to the emergency number stored and a charging port (304) to charge the device.

5. The intelligent digital medicine storage and reminder device (1) as claimed in claim 1, wherein the device (1) being capable to connect with any smart device (1) through the wireless connectivity.

6. The intelligent digital medicine storage and reminder device (1) as claimed in claim 1, wherein said display (200) shows the real-time date and time, patient name, reminding pop-up access, health tips, emergency call service, editing and adding features and alert to the next dose, name of the medicine & doses, schedule time including wireless connectivity, battery (111) status.

7. The intelligent digital medicine storage and reminder device (1) is claim as claimed 1, wherein microcontroller (110) having a weight, pressure sensors, so if containers are achieving movements, the particular movement record and send to the data to server and notify it.

8. The intelligent digital medicine storage and reminder device (1) as claimed in claim 1, wherein said RFID detector (106) identify the user on tapping the RFID card and being capable to determine the time and schedule of the medicine for said user.

9. The intelligent digital medicine storage and reminder device (1) as claimed in claim 1, wherein consumption of medicine by user being capable to track though artificial intelligence based smart camera.
